(19) United States
(12) Patent Application Publication
(10) Pub. No.: US 2003/0211550 A1
(43) Pub. Date: Nov. 13, 2003

(54) Multiplex cytokine analysis

(76) Inventors: Timothy Johann, Sunnyvale, CA (US); Sheila Irao, San Jose, CA (US)

Correspondence Address: RICHARD ARON OSMAN, SCIENCE AND TECHNOLOGY LAW GROUP, 75 DENISE DRIVE, HILLSBOROUGH, CA, 94010

(21) Appl. No.: 10/444,563

(22) Filed: May 23, 2003

Related U.S. Application Data
(63) Continuation of application No. 09/417,292, filed on Oct. 13, 1999, now abandoned.

(51) Int. Cl. G01N 33/53; G01N 33/542; C12M 1/34

(52) U.S. Cl. 435/7.9; 435/287.2

(57) ABSTRACT

The invention provides methods and compositions for the simultaneous, quantitative detection of cytokines in a sample. The methods use a solid phase array comprising a plurality of different antibodies arrayed in corresponding discrete array elements and specific for a corresponding plurality of different cytokines. The invention also provides methods of manufacturing and using the arrays to for the simultaneous, quantitative detection of cytokines in samples.

### CROSS-REFERENCE TO RELATED APPLICATION

This application is a continuation of and claims priority under 35 U.S.C.§ 120 to 09/417,292, filed Oct., 13, 1999, having the same title and inventors, which is incorporated herein by reference.

### INTRODUCTION

1. Field of the Invention

The field of the invention is cytokine analysis.

2. Background

Cytokines are signaling proteins which are important in many diverse fields of research and in the diagnosis and clinical treatment of disease. For example, cytokines regulate the body's inflammatory response to infection. Tumor necrosis factor alpha, interleukin-1, interleukin-6, interleukin-8, and interleukin-12 are pro-inflammatory cytokines which have been implicated with sepsis. Anti-inflammatory cytokines such as interleukin-4 and interleukin-10 are also produced in response to infection to turn down the inflammatory response. Monitoring the serum levels of these and other cytokines can provide a measure of immune activity in the host. For example, several clinical studies have demonstrated a statistical link between TNF-a levels and severity of infection. Furthermore, research has indicated that different pathogens elicit unique patterns of cytokine expression. These data demonstrate that cytokines are excellent molecular markers for the diagnosis of septic inflammatory response.

The current methods of choice for the analysis of cytokine expression in blood samples are quantitative RT-PCR and ELISA. Quantitative RT-PCR measures the level of cytokine mRNA in cells interest. This method requires the extraction of mRNA from cells and thus requires a great deal of pre-assay preparation. The rate determining step of this assay is also the capital intensive step (requiring the expensive RT-PCR instrument) which severely limits scaling up the throughput of this method. Currently, only two cytokines can be detected per assay reaction with quantitative RT-PCR which also limits the potential throughput of this assay. ELISA, on the other hand, directly measures the concentration of cytokine protein in serum or cell supernatants. ELISA assays require no pre-assay preparation. The capital intensive step (detection) is not the longest step in ELISA which allows greater potential throughput than quantitative RT-PCR. These assays do, however, only measure the concentration of one cytokine per reaction which limits their throughput.

The present invention relates to antibody array technology—a powerful tool for the parallel analysis of multiple gene products at the protein level. This technology complements that of DNA microarrays by facilitating the analysis of post transcriptional regulatory events. More particularly, the invention provides ELISA-type assays which analyze the concentrations of multiple proteins such as cytokines per reaction.

### SUMMARY OF THE INVENTION

The invention provides methods and compositions for the simultaneous, quantitative detection of cytokines in a sample. The methods use a solid phase array comprising a plurality of different antibodies arrayed in corresponding discrete array elements and specific for a corresponding plurality of different cytokines. In particular embodiments, the cytokines are selected from GM-CSF, IL1α, IL1β, IL2, IL4, IL6, IL7, IL8, IL10, IL12, TNFα, APO-1, sICAM-1, IFN-α, IFN-γ, IL3, IL5, IL13, IL15, IL16, MCP-1, SAA and sVCAM-1; the plurality is at least five, preferably at least eight, more preferably eleven; the antibodies are monoclonal; each antibody is bound to a different one of the corresponding cytokines; and the array is a microarray on a plastic or glass substrate made by contact deposition. The invention also provides methods of manufacturing and using the arrays for the simultaneous, quantitative detection of cytokines in samples.

### DESCRIPTION OF SPECIFIC EMBODIMENTS OF THE INVENTION

The following descriptions of particular embodiments and examples are offered by way of illustration and not by way of limitation. Unless contraindicated or noted otherwise, in these descriptions and throughout this specification, the terms “a” and “an” mean one or more and the term “or” means and/or.

The methods use solid phase arrays of cytokine-specific antibodies arrayed in corresponding discrete array elements. The array elements are discrete regions of a substrate surface in fluid connection such that all the elements of the array can be incubated, washed, etc. in a single continuous medium. Hence, an array is distinct from assay formats where each specific antibody is separated in discrete, fluid-separated incubation wells as in a microtiter plate.

The antibody arrays may be constructed by a number of methods known in the art on a wide variety of substrates such as glass, silicon, plastics, nylon membranes, etc., including contact deposition, e.g. U.S. Pat. Nos. 5,807,522; 5,770,151, etc.; flow path-based methods, e.g. U.S. Pat. No. 5,384,261; dip-pen nanolithography-based methods, e.g. Piner, et al., Science Jan. 29, 1999: 661-663, etc.; etc. See also copending Ser. No.09/150,502, describing capillary printing systems which may be used for antibody array manufacturing. In a preferred embodiment, the antibodies are arrayed at corresponding discrete elements in high density, i.e. microarrays, generally at least 100, preferably at least 1000, more preferably at least 10,000, most preferably at least 100,000 discrete elements per square centimeter.

The antibodies may be intact or fragments, purified or recombinantly expressed, monoclonal or polyclonal and may be affinity purified. In a particular embodiment, each cytokine-specific antibody of the array is a monoclonal antibody.

The antibodies may be specific for pluralities of a wide variety of cytokines, particularly lymphokines. Suitable cytokines may be purchased in purified or recombinant from commercial sources, expressed from commercially and/or publically available clones, and/or purified from tissues. In a particular embodiment, the cytokines are of native human sequence, though homologs from a wide variety of animal species, particularly mammalian (e.g. murine) species are frequently available and may be used. One unexpected finding of the invention was that a plurality of different cytokines could be simultaneously measured in the same, single incubation, particularly where the plurality is at least five, more particularly at least eight, most particularly at least eleven different cytokines. Exemplary human sequence cytokines shown to be so detectable are shown in Table 1.

TABLE 1
Cytokines shown to be specifically, quantitatively detectable in multiplex
immunoassays.

| | | | | | |
|---|---|---|---|---|---|
| GM-CSF | IL4 | IL10 | sICAM-1¹ | IL5 | MCP-1² |
| IL1α | IL6 | IL12 | IFN-α | IL13 | SAA³ |
| IL1β | IL7 | TNFα | IFN-γ | IL15 | sVCAM-1⁴ |
| IL2 | IL8 | APO-1 | IL3 | IL16 | |

¹Intercellular Adhesion Molecule
²Macrophage Chemoattractant Protein
³Serum Amyloid A
⁴Soluble Vascular Cell Adhesion Molecule

Accordingly, the disclosed arrays may be used to simultaneously, quantitatively detect such cytokines in samples. A wide variety of cytokine containing samples maybe subject to analysis by these methods, e.g. serum, urine, cerebral spinal fluid, etc. Furthermore, unlike PCR-based methods, antibody array technology can measure gene expression in samples which contain no RNA. This greater availability results in the ability to conduct detailed statistical studies with antibodies arrays which are not possible with other assays.

Incubation and wash conditions are readily determined empirically, as shown below, such that the targeted plurality of cytokines may be simultaneously assayed in a single incubation medium. A wide variety of methods may be used to detect the specifically bound cytokine analytes, including directly labeled cytokine specific antibody reagents, sandwich format immunoassays, cytokine-receptor binding assays, etc. Table 2 shows various cytokine pluralities simultaneously assayed by exemplary methods.

TABLE 2
Specific, Quantitative Detection of Cytokines in Micro-Array Immunoassays

| | | | | | Specific, |
|---|---|---|---|---|---|
| | | | | Detection | Quantitiative |
| Exp | Antibody | Specificity | Array | Format | Detection |
| #12 | Mabs | IL1α, IL1β, IL2, | polystyrene, | sandwich assay | +, +, +, +, +, |
| | | IL4, IL12 | low density, | HRP-STR | + |
| | | | contact | | |
| | | | deposition | | |
| #16 | Affinity | IL1α, IL1β, IL2, | glass, high | ³H-labeled | +, +, +, +, +, |
| | purified | IL4, IL6, IL7, | density, contact | cytokine- | +, +, +, + |
| | polyclonals | IL8, IL10, IL12 | deposition | specific | |
| | | | | antibody | |
| #25 | Mabs | GM-SCF, IL1α, | glass, high | sandwich assay, | +, +, +, +, +, |
| | | IL1β, IL2, IL4, | density, contact | HRP-STR | +, +, +, +, +, |
| | | IL6, IL7, IL8, | deposition | | |
| | | IL10, IL12, | | | |
| | | TNFα | | | |
| #27 | Mabs | GM-SCF, IL1α, | nylon, high | sandwich assay, | +, +, +, +, +, |
| | | IL1β, IL2, IL4, | density, ink jet | rhodamine | +, +, +, +, +, |
| | | IL6, IL7, IL8, | deposition | conjugate | + |
| | | IL10, IL12, | | | |
| | | TNFα | | | |
| #33 | Mabs | GM-SCF, IL1α, | polystyrene, | sandwich assay, | +, +, +, +, +, |
| | | IL1β, IL1, IL4, | high density, | HRP-STR | +, +, +, +, +, |
| | | IL6, IL7, IL8, | contact | | + |
| | | IL10, IL12, | deposition | | |
| | | TNFα | | | |
| #51 | Mabs | APO-1, | glass, high | sandwich assay, | +, +, +, +, +, |
| | | sICAM-1, IFN-α, | density, contact | HRP-STR | +, +, +, +, +, |
| | | IFN-γ, IL3, IL5, | deposition | | +, + |
| | | IL13, IL15, IL16, | | | |
| | | MCP-1, SAA, | | | |
| | | sVCAM-1 | | | |
| #55 | Mabs | GM-SCF, TNFα, | glass, high | sandwich assay, | +, +, +, +, +, |
| | | APO-1, | density, contact | HRP-STR | +, +, +, + |
| | | sICAM-1, IFN-α, | deposition | | |
| | | IFN-γ, MCP-1, | | | |
| | | SAA, sVCAM-1 | | | |
| #62 | Mabs | IL1α, IL1β, IL2, | glass, high | sandwich assay, | +, +, +, +, +, |
| | | IL4, IL6, IL7, | density, contact | HRP-STR | +, +, +, +, +, |
| | | IL8, IL10, IL12, | deposition | | +, +, +, + |
| | | IL3, IL5, IL13, | | | |
| | | IL15, IL16. | | | |
| #86 | Mabs | Table 1 cytokines | polystyrene, | sandwich assay, | +, +, +, +, +, |
| | | | high density, | HRP-STR | +, +, +, +, +, |
| | | | contact | | +, +, +, +, +, |
| | | | deposition | | +, +, +, +, +, |
| | | | | | +, +, + |

### EXEMPLARY EXPERIMENTAL PROTOCOLS

Using the following protocols, we constructed a prototype antibody array for the simultaneous detection of 11 different cytokines in serum and cell supernatants. This assay was based on an ELISA sandwich format with chemiluminescent detection. Chemiluminescent detection, as opposed to fluorescent detection, requires no excitation of sample and allows the simultaneous detection of several different arrays at once resulting in a substantial increase in the throughput. Eleven distinct cytokines were successfully simultaneously, quantitatively assayed and the measurements validated by comparison with RT-PCR assays.

Array construction. Capture antibodies (Biosource International) for 11 cytokines (GM-CSF, IL1α, IL1β, IL2, IL4, IL6, IL7, IL8, IL10, IL12 and TNFα) were spotted at a concentration of 0.5 mg/ml directly into the bottom of 12 well plates (Corning) which had been pre-coated for optimal antibody binding. Biotinylated bovine albumin was spotted at a concentration of 100 ug/ml as fiduciary material. Array elements were roughly 1000 microns in diameter with 3000 micron center to center spacing. Once spotted, plates were immediately covered and incubated overnight at 4° C. Arrays were then washed 4× with a solution of PBS (10 mM phosphate, 2.7 mM potassium chloride, 137 mM sodium chloride, pH 7.4) and 0.1% Tween-20 (Sigma) to remove unbound antibody. Plates were then blocked for 2 hours at room temperature with a solution containing 150 mM sodium, 4 mM potassium, 140 mM chloride, 10 mM phosphate (pH 7.4) and 5% BSA.

Assay conditions. The BSA solution was removed from each array followed by 4 washes with a solution of 0.005% tween-20 in PBS. 1 ml of sample was then applied to each array and incubated with shaking (120 rpm) for 2 hours at room temperature. Samples were removed and arrays washed 4× with 0.005% tween-20 in PBS. 1 ml of a solution containing biotinylated antibodies to each of the 11 cytokines (GM-CSF, IL1α, IL1β, IL2, IL4, IL6, IL7, IL8, IL10, IL12 and TNFα) at a concentration of 0.5 ug/ml in 150 mM sodium, 4 mM potassium, 140 mM chloride, 10 mM phosphate (pH 7.4) and 10% BSA was added to each well and incubated with shaking at room temperature for 1 hour. The antibody solution was removed and 1 ml of a horseradish-peroxidase-streptavidin conjugate (HRP-STR), 0.2 ug/ml in HPE-dilution buffer (Research Diagnostics Inc.) was added to each array and incubated for 30 minutes at room temperature with shaking. The HRP-STR solution was removed and arrays washed 4× with 0.005% Tween-20 in PBS solution. SuperSignal™ (Pierce) chemiluminescent substrate was mixed 1:1 with provided peroxide solution, 500 ul of this mixture was added to each array, and the entire plate imaged simultaneously with a peltier cooled CCD camera imaging system (ChemiImager™ 4000, Alpha Innotech Co.). X-Ray film can also be used as a detection system. Data can be quantitated with any convenient image analysis program.

Human THP-1 cells. The human acute monocytic leukemia cell line THP-1 used in this study was maintained at 37° C. in a humidified incubator containing 5% CO₂. These cells were grown in RPMI-1640 medium (Gibco BRL) supplemented with 10% fetal calf serum (Gibco BRL), glucose (4.5 g/l), 5×10⁻⁵ M b-mercaptoethanol, 1 mM sodium pyruvate, streptomycin (100 mg/ml), and penicillin (100 U/ml). The cells were split into 6-well cell culture plates, 5 ml per plate, at a density of 5×10⁵ cells/ml in complete RPMI1640 medium. Before activation of the cells with E. coli or P. aeruginosa as inflammantory stimuli, the cells were treated with 1.6×10⁻⁹ M phorbol-1 2-myristate-13 acetate (PMA) (Sigma) for 48 hr. After this incubation time the cells were treated with different concentrations (1×10¹-10⁵ cells/ml) of exponentially grown E. Coli and P. aeruginosa bacterial cells for 8 hr. Both supernatant fluids and the cell pellet were collected and stored at −80° C. for later cytokine assays and RNA isolation/RT-PCR dectection of cytokine mRNA.

Isolation of RNA. The RNA isolation method with TRIzol Reagent (Gibco BRL) used is essentially as developed by Chomoczynski, P., and N. Sacchi.(1987, Anal. Biochem. 162:156-159). The frozen cell pellets (about 2.5×10⁶ cells) were lysed by resuspending in 1 ml of TRIzol Reagent (Gibco BRL) and repetitive pipetting. 200 ml of chloroform was than added and the samples were mixed vigorously by vortexing for 15 seconds. After centrifugation of the samples at 14,000 rpm for 15 min at 4° C., the colorless upper aqueous phase containing the RNA was transferred into a fresh RNase-free tube. The RNA was precipitated from the aqueous phase by mixing with 0.5 ml of isopropyl alcohol. Samples were incubated at room temperature for 10 min before centrifugation at 4° C. for 7,500 rpm for 10 min. The RNA pellet was washed once with 0.5 ml of 75% ethanol, before allowed to air dry for 10 min. The concentration and purity of the RNA isolated was determined by measuring the OD₂₆₀ in 2 mM Na₂PO₄ (pH8.0) in a spectrophotometer and by agarose gel electrophoresis.

RT-PCR detection of cytokine mRNA. The RT-PCR assay used in this work is essentially as described elsewhere (Murphy et al., 1993, J. Immunol. Methods. 162:211-223). Oligonucleotide primers targeting a 206-bp region of the human IL-6 gene (Zilberstein et al., 1986, EMBO J. 5:2529-37) were used to detect IL-6 specific mRNA in treated THP-1 cells. These IL-6 specific PCR primers were designed based on the NCBI sequence query for the human IL-6 gene (accession number X04430). Oligonucleotide primers targeting a 107-bp region of the human IL-1β gene (Nishida et al., 1987) were used to detect IL-1β specific mRNA in untreated and treated THP-1 cells. These IL-1β specific PCR primers were designed based on the NCBI sequence query for the human IL-1β gene (accession number M15330). All the primers were prepared by Operon (Alameda, Calif.). RT (reverse transcriptase) reactions were carried out in a final volume of 20 ml containing 1 mg of total RNA and the components supplied by PE Applied Biosystems (Foster City, Calif.) in the TaqMan Gold RT-PCR kit. RT reactions were performed as described in the kit protocol. For the following PCR reaction 1 ml of the RT reaction sample was used in a final volume of 30 ml containing all the components supplied by PE Applied Biosystems in the TaqMan Gold RT-PCR kit. PCR reactions were performed as described in the kit protocol. 10 ml of each PCR product was loaded onto a 3% NuSieve/1% SeaKem GTG Agarose gel (FMC Bio-Products, Rockland, Me.) in 1×TBE buffer and subsequent electrophoresis was performed at 200V for 30-45 min.

All publications and patent applications cited in this specification and all references cited therein are herein incorporated by reference as if each individual publication or patent application or reference were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. An array comprising at least five different antibodies arrayed in corresponding discrete array elements and specific for corresponding five different cytokines.

2. An array according to claim 1, comprising at least ten different antibodies arrayed in corresponding discrete array elements and specific for corresponding ten different cytokines.

3. An array according to claim 1, wherein the cytokines are selected from the group consisting of GM-CSF, IL4, IL10, sICAM-1, IL5, MCP-1, IL1α, IL6, IL12, IFN-α, IL13, SAA, IL1β, IL7, TNFα, IFN-γ, IL15, sVCAM-1, IL2, IL8, APO-1, IL3 and IL16.

4. An array according to claim 2, wherein the cytokines are selected from the group consisting of GM-CSF, IL4, IL10, sICAM-1, IL5, MCP-1, IL1α, IL6, IL12, IFN-α, IL13, SAA, IL1β, IL7, TNFα, IFN-γ, IL15, sVCAM-1, IL2, IL8, APO-1, IL3, and IL16.

5. An array according to claim 2, wherein the cytokines are GM-CSF, IL1α, IL1β, IL2, IL4, IL6, IL7, IL8, IL10, IL12 and TNFα.

6. An array according to claim 1, wherein each antibody is specific for and bound to a different one of the corresponding cytokines.

7. An array according to claim 2, wherein each antibody is specific for and bound to a different one of the corresponding cytokines.

8. An array according to claim 3, wherein each antibody is specific for and bound to a different one of the corresponding cytokines.

9. An array according to claim 1, wherein the antibodies are monoclonal.

10. An array according to claim 2, wherein the antibodies are monoclonal.

11. An array according to claim 3, wherein the antibodies are monoclonal.

12. A method for the simultaneous, quantitative detection of cytokines in a sample, the method comprising the step of contacting the sample with an array according to claim 1, whereby at least five different sample cytokines are simultaneously quantitatively measured.

13. A method for the simultaneous, quantitative detection of cytokines in a sample, the method comprising the step of contacting the sample with an array according to claim 2, whereby at least ten different sample cytokines are simultaneously quantitatively measured.

14. A method for the simultaneous, quantitative detection of cytokines in a sample, the method comprising the step of contacting the sample with an array according to claim 3, whereby at least five different sample cytokines corresponding to those of the group consisting of GM-CSF, IL4, IL10, sICAM-1, IL5, MCP-1, IL1α, IL6, IL12, IFN-α, IL13, SAA, IL1β, IL7, TNFα, IFN-γ, IL15, sVCAM-1, IL2, IL8, APO-1, IL3 and IL16 are simultaneously quantitatively measured.

15. A method for the simultaneous, quantitative detection of cytokines in a sample, the method comprising the step of contacting the sample with an array according to claim 4, whereby at least ten different sample cytokines corresponding to those of the group consisting of GM-CSF, IL4, IL10, sICAM-1, IL5, MCP-1, IL1α, IL6, IL12, IFN-α, IL13, SAA, IL1β, IL7, TNFα, IFN-γ, IL15, sVCAM-1, IL2, IL8, APO-1, IL3 and IL16 are simultaneously quantitatively measured.

16. A method for the simultaneous, quantitative detection of cytokines in a sample, the method comprising the step of contacting the sample with an array according to claim 5, whereby sample cytokines GM-CSF, IL1α, IL1β, IL2, IL4, IL6, IL7, IL8, IL10, IL12 and TNFα are simultaneously quantitatively measured.

17. A method for the simultaneous, quantitative detection of cytokines in a sample, the method comprising the step of contacting the sample with an array according to claim 9, whereby at least five different sample cytokines are simultaneously quantitatively measured.

18. A method for the simultaneous, quantitative detection of cytokines in a sample, the method comprising the step of contacting the sample with an array according to claim 10, whereby at least ten different sample cytokines are simultaneously quantitatively measured.

19. A method for the simultaneous, quantitative detection of cytokines in a sample, the method comprising the step of contacting the sample with an array according to claim 11, whereby at least five different sample cytokines corresponding to those of the group consisting of GM-CSF, IL4, IL10, sICAM-1, IL5, MCP-1, IL1α, IL6, IL12, IFN-α, IL13, SAA, IL1β, IL7, TNFα, IFN-γ, IL15, sVCAM-1, IL2, IL8, APO-1, IL3 and IL16 are simultaneously quantitatively measured.

20. An array comprising at least ten different antibodies arrayed in corresponding discrete array elements and specific for corresponding ten different cytokines,
wherein the array is a microarray wherein the elements are arrayed at least 1000 elements per square centimeter,
wherein the cytokines are GM-CSF, IL1α, IL1β, IL2, IL4, IL6, IL7, IL8, IL10 , IL12 and TNFα,
wherein each antibody is specific for and bound to a different one of the corresponding cytokines, and
wherein the antibodies are monoclonal.
